(19) United States
(12) Patent Application Publication
(10) Pub. No.: US 2024/0269331 A1
(43) Pub. Date: Aug. 15, 2024

(54) FLUID-BASED POWDER CONVEYANCE SYSTEMS AND METHODS FOR MEDICAL DEVICE CLEANING AND/OR LUMEN CLEANING

(71) Applicant: Saban Ventures Pty Limited, Macquarie Park, NSW (AU)

(72) Inventors: Gilbert ALEMANA, Jordan Springs, NSW (AU); Ahmed ALI, Dundas Valley, NSW (AU); David PIDCOCK, Dulwich Hill, NSW (AU); Shihan TISSERA, Carlingford, NSW (AU); Herleen RUPRAI, Kellyville, NSW (AU)

(21) Appl. No.: 18/567,224

(22) PCT Filed: Jun. 9, 2022
(86) PCT No.: PCT/AU2022/050569

§ 371 Date: Dec. 5, 2023

(30) Foreign Application Priority Data
Jun. 9, 2021 (AU) 2021901732

(51) Int. Cl. A61L 2/18; A61L 2/20; B08B 9/032

(52) U.S. Cl. CPC A61L 2/18; A61L 2/20; B08B 9/0328; A61L 2202/15; A61L 2202/24

(57) ABSTRACT

Systems and methods that integrate fluid-based powder conveyance for cleaning articles, including lumens and medical devices are presented. For example, a method of cleaning an article comprises a step of flowing a first fluid comprising a cleaning agent to a chamber. Flowing the first fluid comprising the cleaning agent to the chamber creates a pressure differential across a filter fluidly coupled to the chamber. When a pressure differential reaches a threshold, indicating that the cleaning agent has been accurately metered, flow of the first fluid comprising the cleaning agent is stopped. Next, flowing a second fluid through the chamber conveys the cleaning agent to an article, e.g., through a lumen of a medical or culinary device, thereby cleaning the article.

### CROSS REFERENCE TO RELATED APPLICATIONS

This application claims the benefit of Australian provisional application number 2021901732, filed on Jun. 9, 2021, the entirety of which is incorporated by reference herein.

### BACKGROUND

Any discussion of the prior art throughout the specification should in no way be considered as an admission that such prior art is widely known or forms part of common general knowledge in the field.

There are numerous challenges in maintaining the cleanliness and sterility of various articles including ice machines, soda machines, coffee/espresso machines, and medical devices using cleaning devices, cleaning agents, and methods that preserve the food/medical safety of the article. Often the difficult of cleaning such articles arises from the various tubing/channels that are difficult to access or clean using unspecialized equipment.

For example, an endoscope is an elongate tubular medical device that may be rigid or flexible and which incorporates an optical or video system and light source. Typically, an endoscope is configured so that one end can be inserted into the body of a patient via a surgical incision or via one of the natural openings of the body. Internal structures near the inserted end of the endoscope can thus be viewed by an external observer.

As well as being used for investigation, endoscopes are also used to carry out diagnostic and surgical procedures. Endoscopic procedures are increasingly popular as they are minimally invasive in nature and provide a better patient outcome (through reduced healing time and exposure to infection) enabling hospitals and clinics to achieve higher patient turnover.

Endoscopes typically take the form of a long tube-like structure with a ‘distal tip’ at one end for insertion into a patient and a ‘connector end’ at the other end, with a control handle at the center of the length. The connector end is normally hooked up to a supply of light, water, suction and pressurized air. The control handle is held by the operator during the procedure to control the endoscope via valves and control wheels. The distal tip contains the camera lens, lighting, nozzle exits for air and water, exit point for suction and forceps. All endoscopes have internal channels used either for delivering air and/or water, providing suction or allowing access for forceps and other medical equipment required during the procedure. Some of these internal channels run from one end of the endoscope to the other, while others run via valve sockets at the control handle. Some channels bifurcate while and others join from two into one.

The high cost of endoscopes means they must be re-used. As a result, because of the need to avoid cross infection from one patient to the next, each endoscope must be thoroughly cleaned and disinfected or sterilized after each use. This involves the cleaning of not only the outer of the endoscope, but also cleaning and disinfecting the internal channels/lumens. The high cost of endoscopes means they must be re-used. As a result, because of the need to avoid cross infection from one patient to the next, each endoscope must be thoroughly cleaned and disinfected or sterilized after each use. This involves the cleaning of not only the outer of the endoscope, but also cleaning and disinfecting the internal channels/lumens.

Endoscopes used for colonoscopy procedures are typically between 2.5 and 4 meters long and have one or more lumen channels of diameter of no more than a few millimeters. Ensuring that such long narrow channels are properly cleaned and disinfected between patients presents a considerable challenge. The challenge of cleaning is also made more difficult by the fact that there is not just one configuration/type of endoscope. Indeed, there are a variety of endoscopic devices, each suited to a particular insertion application i.e. colonoscopes inserted into the colon, bronchoscopes inserted into the airways and gastroscopes for investigation of the stomach. Gastroscopes, for instance, are smaller in diameter than colonoscopes; bronchoscopes are smaller again and shorter in length while duodenoscopes have a different tip design to access the bile duct.

A variety of options are available to mechanically remove biological residues from the lumen which is the first stage in the cleaning and disinfection process. By far the most common procedure for cleaning the lumens utilize small brushes mounted on long, thin, flexible lines. Brushing is the mandated means of cleaning the lumen in some countries. These brushes are fed into the lumens while the endoscope is submerged in warm water and a cleaning solution. The brushes are then pushed/pulled through the length of the lumens in an effort to scrub off the soil/bio burden. Manual back and forth scrubbing is typically required. Water and cleaning solutions are then flushed down the lumens. These flush-brush processes are repeated three times or until the endoscope reprocessing technician is satisfied that the lumen is clean. At the end of this cleaning process air is pumped down the lumens to dry them. A flexible pull-through device having wiping blades may also be used to physically remove material. A liquid flow through the lumen at limited pressure can also be used.

In general, however, only the larger suction/biopsy lumens can be cleaned by brushing or pull-throughs. Air/water channels are too small for brushes so these lumens are usually only flushed with water and cleaning solution.

After mechanical cleaning, a chemical clean is carried out to remove the remaining biological contaminants. Because endoscopes are sensitive and expensive medical instruments, the biological residues cannot be treated at high temperatures or with strong chemicals. For this reason, the mechanical cleaning needs to be as thorough as possible. In many cases, the current mechanical cleaning methodologies fail to fully remove biofilm from lumens, particularly where cleaning relies on liquid flow alone. Regardless of how good the conventional cleaning processes are, it is almost inevitable that a small microbial load will remain in the channel of the lumen.

There has been significant research to show that the method of cleaning with brushes, even when performed as prescribed, does not completely remove biofilm in endoscope lumens. As well as lacking in efficacy, the current manual brushing procedures suffer from other drawbacks. The large number of different endoscope manufacturers and models results in many minor variations of the manual cleaning procedure. This has led to confusion and ultimately poor compliance in cleaning processes. The current system of brushing is also hazardous in that the chemicals that are currently used to clean endoscopes can adversely affect the reprocessing staff.

The current system of manual brushing is also labor intensive, leading to increased cost. Thus, the current approaches to cleaning and disinfecting the lumens in medical cleaning apparatus are still inadequate and residual microorganisms are now recognized as a significant threat to patients and staff exposed to these devices.

There is evidence of bacterial transmission between patients from inadequate cleaning and disinfection of internal structures of endoscopes which in turn has led to patients acquiring mortal infections. Between 2010 and 2015 more than 41 hospitals worldwide, most in the U.S., reported bacterial infections linked to the scopes, affecting 300 to 350 patients (http://www.modernhealthcare com/article/20160415/NEWS/160419937). It would be expected that a reduction in the bioburden in various medical devices would produce a concomitant overall reduction in infection rates and mortality. In addition, if endoscopes are not properly cleaned and dried, biofilm can build up on the lumen wall. Biofilms start to form when a free-floating microorganism attaches itself to a surface and surrounds itself with a protective polysaccharide layer. The microorganism then multiplies, or begins to form aggregates with other microorganisms, increasing the extent of the polysaccharide layer. Multiple sites of attachment can in time join up, forming significant deposits of biofilm. Once bacteria or other microorganisms are incorporated in a biofilm, they become significantly more resistant to chemical and mechanical cleaning than they would be in their free-floating state. The organisms themselves are not inherently more resistant, rather, resistance is conferred by the polysaccharide film and the fact that microorganisms can be deeply embedded in the film and isolated from any chemical interaction. Any residual biofilm remaining after an attempt at cleaning quickly returns to an equilibrium state and further growth of microorganisms within the film continues. Endoscopes lumens are particularly prone to biofilm formation. They are exposed to significant amounts of bioburden, and subsequent cleaning of the long narrow lumens is quite difficult due to inaccessibility and the inability to monitor the cleaning process.

There is considerable pressure in medical facilities to reprocess endoscopes as quickly as possible. Because endoscopes are cleaned by hand, training and attitude of the technician are important in determining the cleanliness of the device. Residual biofilm on instruments can result in a patient acquiring an endoscope acquired infection. Typically, these infections occur as outbreaks and can have fatal consequences for patients.

There remains a need to overcome or ameliorate at least one of the disadvantages of the prior art, or to provide a useful alternative.

### BRIEF SUMMARY

According to a first aspect of the present invention, there is provided a method of cleaning a lumen comprising:
flowing a first fluid comprising a cleaning agent to a chamber,
wherein flowing the first fluid comprising the cleaning agent to the chamber creates a pressure differential across a filter fluidly coupled to the chamber;
reaching a threshold by the pressure differential, thereby triggering the flowing of the first fluid comprising the cleaning agent to the chamber to stop; and
flowing a second fluid through the chamber to convey the cleaning agent through a lumen, thereby cleaning the lumen.

According to a second aspect of the present invention, there is provided a method of cleaning an article comprising:
flowing a first fluid comprising a cleaning agent to a chamber, wherein flowing the first fluid comprising the cleaning agent to the chamber creates a pressure differential across a filter fluidly coupled to the chamber;
reaching a threshold by the pressure differential, thereby triggering the flowing of the first fluid comprising the cleaning agent to the chamber to stop; and
flowing a second fluid through the chamber to convey the cleaning agent to the article, thereby cleaning the article.

According to a third aspect of the present invention, there is provided a method of cleaning a medical device comprising:
flowing a first fluid comprising a cleaning agent to a chamber,
wherein flowing the first fluid comprising the cleaning agent to the chamber creates a pressure differential across a filter fluidly coupled to the chamber;
reaching a threshold by the pressure differential, thereby triggering the flowing of the first fluid comprising the cleaning agent to the chamber to stop; and
flowing a second fluid through the chamber to convey the cleaning agent to the medical device, thereby cleaning the medical device.

According to a fourth aspect of the present invention, there is provided a method of cleaning a medical device having a lumen comprising:
delivering a target dosage of a cleaning agent to an eductor, wherein the cleaning agent is pneumatically delivered through a filter to achieve the target dosage;
delivering a fluid to the eductor; and
delivering an aggregate of the fluid and the target dosage of the cleaning agent to at least a portion of the lumen.

According to a fifth aspect of the present invention, there is provided a method of cleaning a lumen of a medical device comprising:
dosing a first portion of cleaning agent using a pressure differential across a dosing filter;
mixing/combining/aggregating the first portion of cleaning agent and a first portion of water to form a mixture/combination/aggregate;
injecting the mixture/combination/aggregate into the lumen of the medical device; and
injecting a quantity of air into the lumen of the medical device after the mixture/combination/aggregate.

According to a sixth aspect of the present invention, there is provided a method of cleaning a lumen of a medical device comprising:
providing a device for cleaning the medical device configured to:
dose a first portion of cleaning agent using a pressure differential across a dosing filter;
combine/mix/aggregate the first portion of cleaning agent and a first portion of water to form a mixture;
inject a portion of the mixture into the lumen of the medical device; and
inject a quantity of air into the lumen of the medical device after the portion of the mixture.

According to a seventh aspect of the present invention, there is provided A system for cleaning a medical device having a lumen comprising:
an eductor comprising a filter;
a pneumatic delivery subsystem configured to deliver a cleaning agent to the eductor and comprising a pressure differential mechanism, wherein the pressure differential mechanism is configured to pneumatically the deliver cleaning agent to the eductor when the pressure differential is less than a threshold value;
a liquid delivery subsystem configured to deliver the liquid to the eductor; and
an engine configured to dynamically mix/combine/aggregate the cleaning agent and the liquid and propel the resulting mixture/combination/aggregate through a lumen of a medical device.

Therefore, systems and methods of cleaning an article comprise steps of flowing a first fluid comprising a cleaning agent to a chamber. The inventors contemplate that suitable cleaning agents comprise powders including, without limitation, sodium bicarbonate, sodium chloride, sodium sulfate, glycine, erythritol and mixtures thereof. When the first fluid comprising the cleaning agent flows to the chamber, it encounters a filter fluidly coupled to the chamber. The filter retains cleaning agent based on the pore size of the filter and the particle size distribution of the cleaning agent, creating a pressure differential across the filter. The pressure differential may be monitored continuously, intermittently, and/or by an operator. When the pressure differential reaches a threshold, reaching the threshold triggers the stopping of the delivery of the cleaning agent to the chamber. Advantageously, use of the filter and pressure drop can enable accurate and precise metering of the cleaning agent without the use of in-line weight, volume, or time-based meters. Elimination of such meters obviates the need to clean and/or repair moving parts of the meters that can become fouled or damaged by the cleaning agent. Then a second fluid flows through the chamber to convey the cleaning agent to the article, thereby cleaning the article. As can be appreciated the first fluid and the second fluid may be the same or different.

In some embodiments, the first fluid comprises a first gas. Exemplary first gasses comprise air, nitrogen, argon, carbon dioxide, or mixtures thereof. In one embodiment, the first gas is treated to reduce the water content of the first gas.

In other embodiments, the first fluid comprises a first liquid, and the cleaning agent is substantially insoluble (e.g., having a solubility of less than about 5% by w/w) in the first liquid. For example, ethanol, acetone, and methanol could be used as the first fluid with a sodium bicarbonate cleaning agent, because the sodium bicarbonate may be insoluble in ethanol, and has a solubility of 0.02% w/w in acetone and 2.13% w/w in methanol. Advantageously, the first fluid passes through the filter and the cleaning agent is retained by the filter in the chamber. In exemplary embodiments, the first liquid is an aqueous liquid, an alcohol, a hydrocarbon, carbon dioxide, or mixtures thereof. The first liquid may comprise a detergent. Suitable detergents may comprise surfactants, such as low-foaming, non-ionic, low viscosity liquids at room temperature, water soluble, and/or have a good cleaning power in cold and warm water (15-40° C.) s described in greater detail below.

It can be appreciated that a cross-sectional area of the filter may be positioned perpendicular to the direction of the first fluid flow or at an angle. In some embodiments, the filter is parallel to the direction of the first fluid flow, e.g., positioned on a side of the chamber such that the first fluid makes turns to pass through the filter.

In some embodiments, the second fluid comprises a second gas. Suitable second gasses comprise air, nitrogen, argon, carbon dioxide, or mixtures thereof. The first gas and the second gas may be the same or different. In some embodiments, the second gas comprises a sterilizing gas, e.g., ethylene oxide gas, vaporized hydrogen peroxide, chlorine dioxide gas, vaporized peracetic acid, nitrogen dioxide, or other such gasses.

In other embodiments, the second fluid comprises a second liquid. For example, the second liquid comprises an aqueous liquid, an alcohol, a hydrocarbon, or carbon dioxide. The second liquid may comprise a detergent, such as a surfactant. Suitable surfactants may comprise low-foaming, non-ionic, low viscosity liquids at room temperature, water soluble, and/or have a good cleaning power in cold and warm water (15-40° C.) s described in greater detail below. In some embodiments, the second fluid comprises a disinfectant such as hydrogen peroxide, an antimicrobial, and/or an alcohol.

The disclosure further encompasses systems configured to perform these methods.

Advantageously, the article may comprise a lumen (e.g., the lumen of an endoscope), a medical device, an ice machine (e.g., the conduits and surfaces of an ice machine), a soda dispenser, or other article that would benefit from cleaning via the systems and methods disclosed herein.

Therefore, cleaning systems with integrated fluid-based powder conveyance subsystems and methods that employ on-demand preparation of cleaning solutions comprising a cleaning agent to improve the efficacy of endoscope cleaning in accordance with embodiments of the invention are described. Moreover, the described techniques may be implemented in a variety of medical devices having lumens. The systems and methods described herein can facilitate the use of a bulk reservoir/multi-dose consumable system that is easily accessible to operators. In many embodiments, cleaning systems with integrated fluid-based powder conveyance subsystems can use an integrated fluid-based powder conveying system (positive pressure and/or vacuum) to transfer dry cleaning agent, e.g., sodium bicarbonate powder, from a bulk reservoir to a dosing unit/doser of individual cleaning engines. This can be done on-demand. Fluid-based powder conveying can provide benefits over a mechanical conveying system (e.g., auger/conveyor/gravity feed) as there may be relatively fewer moving parts in the powder path. This can be desirable as many moving parts are susceptible to degradation with exposure to dry solid powder. Another benefit of fluid-based powder conveying is that it can facilitate a variety of arrangements of the bulk reservoir/consumable with respect to the dosing units/engines, both in distance and relative location. This can allow the location of the bulk reservoir/consumable to be optimized for operator access in the device.

Cleaning systems and methods in accordance with the disclosure also employ a filter to facilitate accurate dosing (±5%) of dry cleaning agent, e.g., sodium bicarbonate powder, in a compact (desktop scale) device. Contemplated cleaning systems and methods can achieve this advantage by using a dosing unit/doser comprising a filter on each cleaning engine to repeatedly meter an accurate amount (±5%) of cleaning agent, e.g., sodium bicarbonate, to the eductor. This level of accuracy may be critical to the application as dosing too much cleaning agent, e.g., sodium bicarbonate, to the eductor can risk blocking the internal fluidics of the endoscope and too little can reduce the cleaning efficacy of the system below target levels. Conventional measuring systems for dry powder include time based and volumetric dosing, however these have not yielded the accuracy and precision desirable for this application. Alternatively, gravimetric systems are sufficiently accurate but impractical in size, mechanical complexity and cost for this application.

Accordingly, dosing units in accordance with several embodiments can comprise a particle filter cartridge coupled to the fluid-based powder conveying system. As a cleaning agent, e.g., sodium bicarbonate powder, is retained within the filter (the pore size of the filter is smaller than the majority of the cleaning agent, e.g., sodium bicarbonate powder particles) the differential pressure across the cartridge can increase. The differential pressure measurement can be correlated to the amount of retained cleaning agent, e.g., sodium bicarbonate, thus functioning as a simple yet accurate dry powder metering system.

One embodiment of a method of cleaning a medical device having a lumen comprises: delivering a fluid-based powder cleaning agent (e.g., pneumatically) through a filter to an eductor to achieve a target dosage; delivering a fluid to the eductor; and delivering an aggregate of the fluid and the target dosage of the cleaning agent to at least a portion of the lumen. The delivery of the aggregate can be implemented in any suitable way. For example, a carrier fluid may be used to deliver the aggregate to the lumen. For example, air may be used as the carrier fluid. In another embodiment, the method is repeated iteratively to clean the at least a portion of the lumen. In a further embodiment, the method comprises a step of delivering a surfactant to the eductor, such that the aggregate delivered to at least a portion of the lumen comprises the target dosage of cleaning agent, the fluid, and the surfactant.

Suitable cleaning agents may comprise water soluble, biocompatible powders having a Mohs hardness of about 1 to about 5, or about 2 to about 3. In some embodiments biocompatible powders are those found in the human body or used as food ingredients to reduce health risks should residual cleaning agent remain after a cleaning cycle. Exemplary cleaning agents comprise sodium bicarbonate, sodium chloride, sodium sulfate, glycine, erythritol and mixtures thereof. Any suitable cleaning agent particle sizes may be employed depending on the dosing filter and diameter of the medical device lumen(s). In some embodiments, suitable salts yield aqueous solutions having a pH of about 5 to about 9 to reduce the risk of corrosion.

In another embodiment, a proportion of the first portion of cleaning agent, e.g., sodium bicarbonate to the first portion of water is about from about 0.5% to about 5%; in some embodiments, a proportion of the first cleaning agent to the liquid is about 1% to about 3%. Surprisingly, abrasiveness may be maintained while even though the concentration is below the saturation point. This can be a function of delivering the mixture at high velocity, and the “on-demand” combination of cleaning agent and fluid does not give the mixture enough time for the cleaning agent to dissolve. A further advantage of employing a cleaning agent concentration below the saturation point is that the risk of blockage is reduced. The use of mixtures in which the first portion of cleaning agent is above the saturation point is not excluded. The first portion of cleaning agent, e.g., sodium bicarbonate, may comprise about 1 g to about 10 g or about 4 g to about 6 g. The first portion of water is about 50 g to about 500 g or about 100 g to about 400 g. In one embodiment, air used to flow the cleaning agent from a storage unit to a mixing chamber. It should be appreciated that a dose of cleaning agent, e.g., sodium bicarbonate can be controlled using such air flows.

Advantageously, the systems and methods disclosed herein can achieve effective cleaning at ambient temperature (e.g., about 15° C. to about 25° C.). In one embodiment, the method further comprises a step of heating the water to a temperature before the mixing step. The water may be heated to a temperature that is up to about 40° C.

Surfactants can be included in the cleaning mixture to dissolve and/or loosen bioburden, including biofilms. In one embodiment, the mixing step optionally includes mixing a first portion of surfactant and the first portion of cleaning agent, e.g., sodium bicarbonate, and the first portion of water to form the mixture. Suitable surfactants may be, without limitation, low-foaming, non-ionic, low viscosity liquids at room temperature, water soluble, and/or have a good cleaning power in cold and warm water (15-40° C.). For example alcohol ethoxylates, alcohol alkoxylates, alkyl polyglucosides, and mixtures thereof may be employed in the systems and methods disclosed herein. Blends of surfactants within these groups are also contemplated and may also be used to clean a broader soil range. Some surfactant formulations could also contain other additives to improve its stability, reduce corrosion in device when dosing a concentrated corrosive active for dilution and reduce microbial contamination. Low levels of foaminess can advantageously can allow the mixture to develop kinetic energy that may be impeded by higher levels of foaminess. Non-ionic surfactants can be compatible with other components of the mixture including water soluble cleaning agent salts, e.g., sodium bicarbonate. Low viscosity can facilitate dosing. Water solubility reduces residual risk. Good cleansing power may be characterized by the ability to solubilize, suspend, emulsify soils and/or reduce surface/interfacial tension. The first portion of surfactant can be about 0.1-1% w/w.

Under contemplated pressures and fluid velocities, a flow of the portion of the mixture may be turbulent. In further aspects of the disclosure, the method steps may be repeated such that the method alternates between mixture preparation and cleaning phases to increase the efficiency of the cleaning systems and methods.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

The above embodiments are exemplary only. Other embodiments as described herein are within the scope of the disclosed subject matter.

### BRIEF DESCRIPTION OF THE SEVERAL VIEWS OF THE DRAWINGS

So that the manner in which the features of the disclosure can be understood, a detailed description may be had by reference to certain embodiments, some of which are illustrated in the accompanying drawings. It is to be noted, however, that the drawings illustrate only certain embodiments and are therefore not to be considered limiting of its scope, for the scope of the disclosed subject matter encompasses other embodiments as well. The drawings are not necessarily to scale, emphasis generally being placed upon illustrating the features of certain embodiments. In the drawings, like numerals are used to indicate like parts throughout the various views, in which:

FIG. 1A shows a flow diagram for one embodiment of a method of cleaning an article, in accordance with one or more aspects set forth herein.

FIG. 1B shows a flow diagram for one embodiment of a method of cleaning the lumen of a medical device, in accordance with one or more aspects set forth herein.

FIG. 2 illustrates a block diagram of one embodiment of a cleaning system with an integrated fluid-based powder conveyance subsystem, in accordance with one or more aspects set forth herein.

FIG. 3A shows a block diagram of another embodiment of a cleaning system with an integrated fluid-based powder conveyance subsystem, in accordance with one or more aspects set forth herein.

FIG. 3B shows a block diagram of another embodiment of a cleaning system with an integrated fluid-based powder conveyance subsystem, in accordance with one or more aspects set forth herein.

FIG. 4A shows a block diagram of one embodiment of a cleaning system with an integrated fluid-based powder conveyance subsystem, in accordance with one or more aspects set forth herein.

FIG. 4B shows a block diagram of one embodiment of a cleaning system with an integrated fluid-based powder conveyance subsystem, in accordance with one or more aspects set forth herein.

FIGS. 5A and 5B illustrate one embodiment of a consumable interface module, in accordance with one or more aspects set forth herein.

FIGS. 6A and 6B illustrate one embodiment of an intake manifold module, in accordance with one or more aspects set forth herein.

FIGS. 7A-7C illustrate one embodiment of an engine assembly, in accordance with one or more aspects set forth herein.

FIGS. 8A and 8B illustrate one embodiment of an eductor assembly, in accordance with one or more aspects set forth herein.

FIGS. 9A and 9B illustrate one embodiment of a system for cleaning the lumens of medical devices, in accordance with one or more aspects set forth herein.

FIG. 10 illustrates a consumable interface module that may be implemented in accordance with one or more aspects set forth herein.

Corresponding reference characters indicate corresponding parts throughout several views. The examples set out herein illustrate several embodiments, but should not be construed as limiting in scope in any manner.

### DETAILED DESCRIPTION

The present disclosure relates to fluid-based powder conveying systems and methods in cleaning applications, including medical device reprocessing.

One embodiment of a method of cleaning a medical device having a lumen comprises: delivering a fluid-based powder cleaning agent (e.g., pneumatically) to a chamber through a filter to collect a target dosage of cleaning agent on the filter; delivering a fluid to the chamber; and delivering an aggregate of the fluid and the target dosage of the cleaning agent to at least a portion of the lumen. In another embodiment, the method is repeated iteratively to clean the at least a portion of the lumen. In a further embodiment, the method comprises a delivering a surfactant to the chamber, such that the aggregate delivered to at least a portion of the lumen comprises the target dosage of cleaning agent, the fluid, and the surfactant.

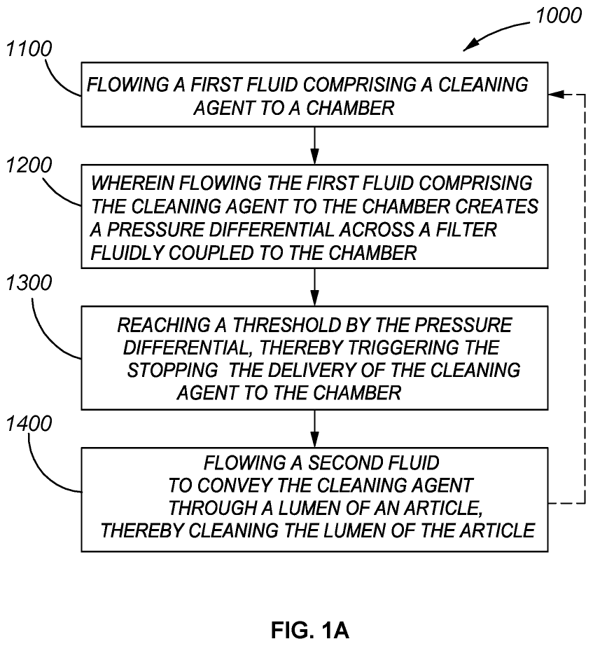

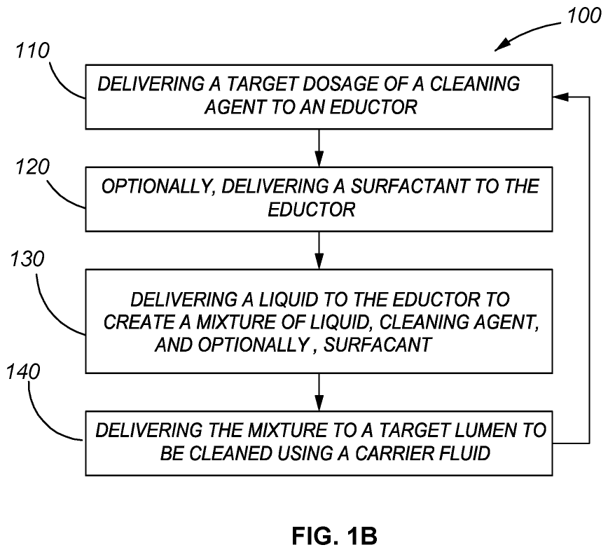

FIG. 1A depicts a flow diagram 1000 for one embodiment of a method of cleaning an article comprises step 1100 of flowing a first fluid comprising a cleaning agent (e.g., sodium bicarbonate) to a chamber. In step 1200 flowing the first fluid comprising the cleaning agent creates a pressure differential across the filter fluidly coupled to the chamber. Once the pressure differential reaches a threshold (e.g., about 20 psi or about 30 psi) in step 1300, delivery of the cleaning agent to the chamber is stopped. Then in step 1400 a second fluid, which may be the same as the first fluid, flows through the chamber to convey the cleaning agent through a lumen of the article, thereby cleaning the lumen of the article. Optionally, the method/process may be repeated to achieve better cleaning of the lumen of the article. As discussed above, contemplated articles include medical devices (e.g., endoscopes) and various kitchen equipment (e.g., ice machines, coffee/espresso makers, soda machines and others).

FIG. 1B illustrates a flow diagram 100 for one embodiment of a method of cleaning the lumen of a medical device, in accordance with one or more aspects set forth herein. In particular, the method 100 includes delivering a target dosage of a cleaning agent 110, e.g., sodium bicarbonate powder, to an eductor. The delivery 110 can be achieved in any of a variety of ways in accordance with embodiments of the invention. For example, in many embodiments, sodium bicarbonate powder is conveyed (e.g., pneumatically) to the eductor. As will be described in further detail below, a filter can be used to achieve a target dosage for the cleaning agent. Note that any of a variety of suitable cleaning agents can be used in accordance with embodiments of the invention. In addition to sodium bicarbonate, suitable cleaning agents can comprise water soluble, biocompatible powders having a Mohs hardness of about 1 to about 5, or about 2 to about 3. In some embodiments biocompatible salts can include sodium bicarbonate, sodium chloride, sodium sulfate, glycine, erythritol and mixtures thereof. Depending on the dosing filter pore size and diameter of the medical device lumen(s), any suitable cleaning agent particle sizes may be employed (e.g., D10 (22 μm), D50 (77 μm), D90 (150 μm)). To reduce the risk of corrosion, the pH of salt solutions can range from about 5 to about 9.

Suitable cleaning agent concentrations can range from about 0.5% to about 5%, or about 1% to about 3% (w/w). The use of mixtures in which the cleaning agent is above its saturation point (e.g., ≥ about 10%) is also contemplated. The first portion of cleaning agent (or target dosage), e.g., sodium bicarbonate, may comprise about 1 g to about 10 g or about 4 g to about 6 g. The first portion of water is about 50 g to about 500 g or about 100 g to about 400 g. In one embodiment, air used to flow the cleaning agent from a storage unit to a mixing chamber.

Optional step 120 comprises delivering a surfactant, e.g., alcohol ethoxylates, alcohol alkoxylates, alkyl polyglucosides, and mixtures thereof, to the eductor. Contemplated surfactants include low-foaming, non-ionic surfactants that can be low viscosity liquids at room temperature, water soluble, and/or have a good cleaning power in cold and warm water (16-40° C.).

Step 130 comprises delivering a liquid to the eductor to create a mixture of liquid, cleaning agent, and optionally, surfactant. For example in many embodiments, water is used to create the mixture/combination/aggregate. As can be appreciated, steps 110, 120, and 130 can be performed in any order to create the aforementioned mixture/combination/aggregate.

The method 100 further includes delivering 140 the mixture to a target lumen to be cleaned. In many embodiments, air is used as the carrier fluid. But any suitable carrier fluid may be used in accordance with embodiments of the invention. Under contemplated pressures and fluid velocities, a flow of the portion of the mixture may be turbulent. As illustrated, method 100 may be repeated for a preset number of cycles, e.g., 20 cycles, or until the lumen(s) of the medical device are clean. Surfactant may be intermittently included across the cycles.

It should be appreciated the illustrated and described method can be implemented in any of a variety of ways in accordance with embodiments of the invention. For example, two or more engines can perform the method steps in continuous, alternating cycles such that one engine performs the ‘dosing steps’ (e.g. the delivery of the constituent components for mixing) while the other engine propels the mixture/combination/aggregate through the lumen (the cleaning phases), and then the two engines can switch roles, thereby increasing the efficiency of the cleaning systems and methods as compared to performing the dosing and cleaning phases in series, because the dosing step can be slow.

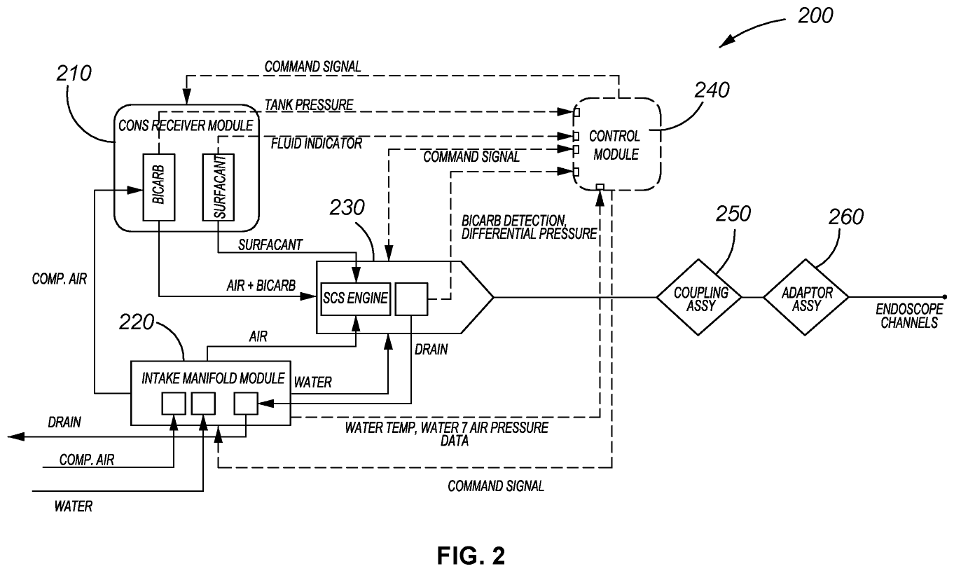

Moreover, the above described and illustrated method can be implemented using any of a variety of system configurations. Thus, for example, FIG. 2 illustrates a block diagram of one embodiment of cleaning system with an integrated fluid-based powder conveyance subsystem 200 that includes consumable receiver module 210, intake manifold module 220, cleaning engine 230, control module 240, coupling assembly 250, and adaptor assembly 260. The consumable receiver module 210 may be configured to receive a cleaning agent for use in cleaning a target lumen. In some embodiments, the receiver module 210 may further be configured to receive surfactant, which can enhance the cleaning of the target lumen as discussed previously. The intake manifold module 220 may supply air/water to create the mixture and/or serve as the carrier fluid. Of course, any suitable fluids may be implemented in accordance with embodiments of the invention. For example, in some embodiments, gaseous nitrogen may be used to propel a cleaning mixture through the lumen. The cleaning engine 230 may comprise an eductor, within which the cleaning mixture is created and carried from to the target lumen. The control module 240 may be used to control the operation of the system 200. Coupling assembly 250 may be used to facilitate the coupling of the system to a medical device; and an adaptor assembly 260 may be used to facilitate the coupling to specific brands/models of medical devices.

In one embodiment, engine 230 receives the first portion of cleaning agent, e.g., sodium bicarbonate, from consumable receiver module 210 and water from intake manifold module 220, and a mixture/combination/aggregate can be formed therefrom.

In one embodiment, a dosing filter (not illustrated) controls the proportion of the first portion of cleaning agent, e.g., sodium bicarbonate, to the first portion of water is about 0.5% to about 5%, or about 1% to about 3% w/w. The first portion (or target dosage) of cleaning agent, e.g., sodium bicarbonate, may comprise about 1 g to about 10 g or about 4 g to about 6 g. The first portion of water is about 50 g to about 500 g or about 100 g to about 400 g. In one embodiment, air used to flow the cleaning agent, e.g., sodium bicarbonate, from a storage unit to a mixing chamber. It should be appreciated that a dose of cleaning agent, e.g., sodium bicarbonate, can be controlled using such air flows.

In some embodiments, the first portion of cleaning agent, e.g., sodium bicarbonate, to the first portion of water in the mixture is above the saturation point of cleaning agent in water.

In one embodiment, intake manifold module 220 heats the water up to about 40° C. before the mixing step. Suitable water temperatures include ambient temperatures, e.g., about 15° C. to about 25° C.

In one embodiment, engine 230 also receives surfactant from consumable receiver module 210 and mixes a first portion of surfactant and the first portion of cleaning agent, e.g., sodium bicarbonate, and the first portion of water to form the mixture. The first portion of surfactant can be about 0.1 g to about 3 g or about 0.5 g to about 1.5 g. Suitable surfactants comprise alcohol ethoxylates, alcohol alkoxylates, and/or alkyl polyglucosides, without limitation.

One having ordinary skill in the art would appreciate that the air and water pressures may be selected to propel the mixture, or a portion thereof, and the quantity of air at velocities that result in turbulent flow of the portion of the mixture. Contemplated internal diameters of lumens range from about 0.9 mm to about 6.0 mm.

Although, one configuration has been illustrated, it should be clear that systems for cleaning medical devices having lumens can be implemented using any of a variety of configurations according to embodiments of the invention.

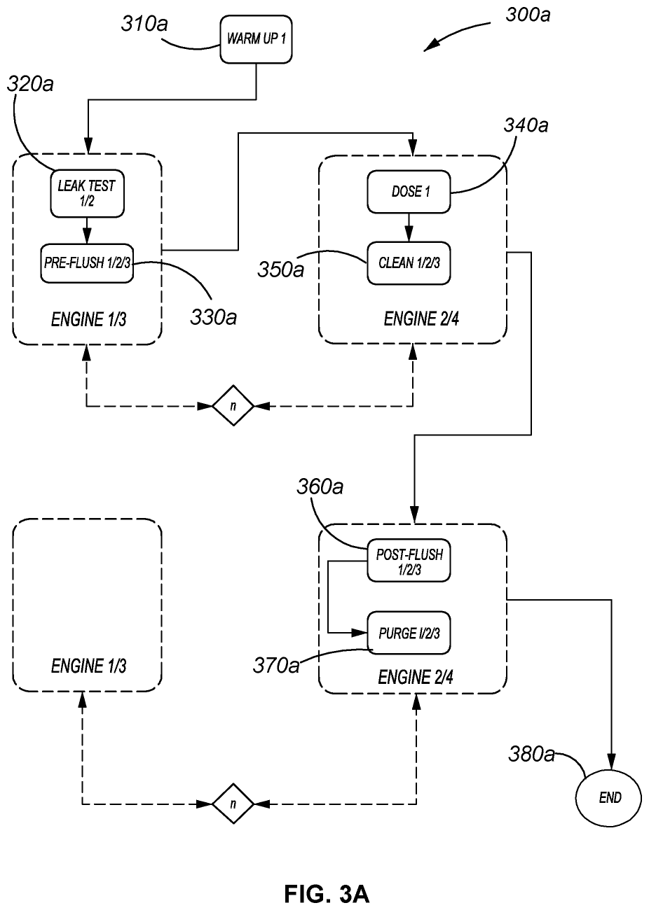

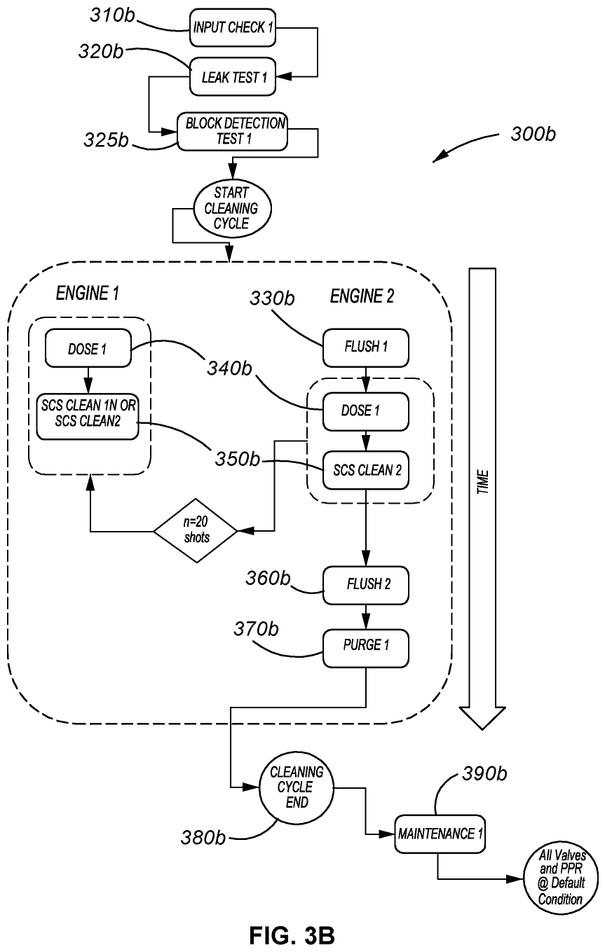

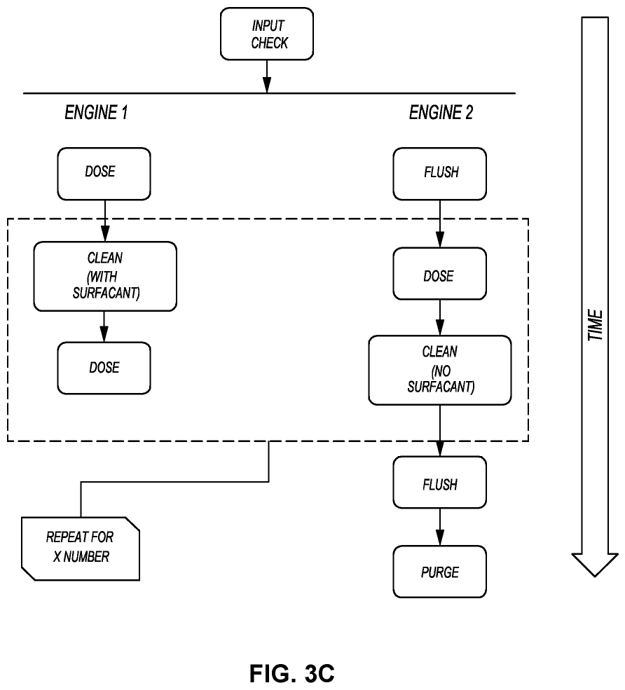

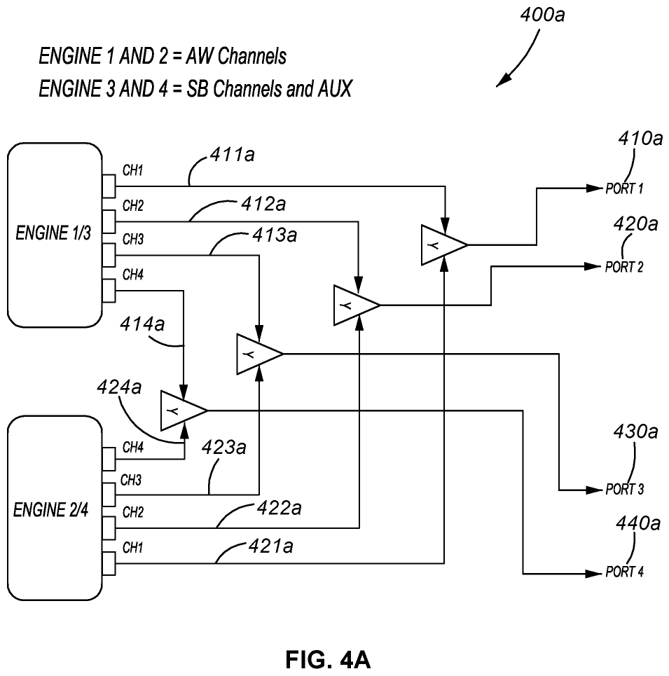

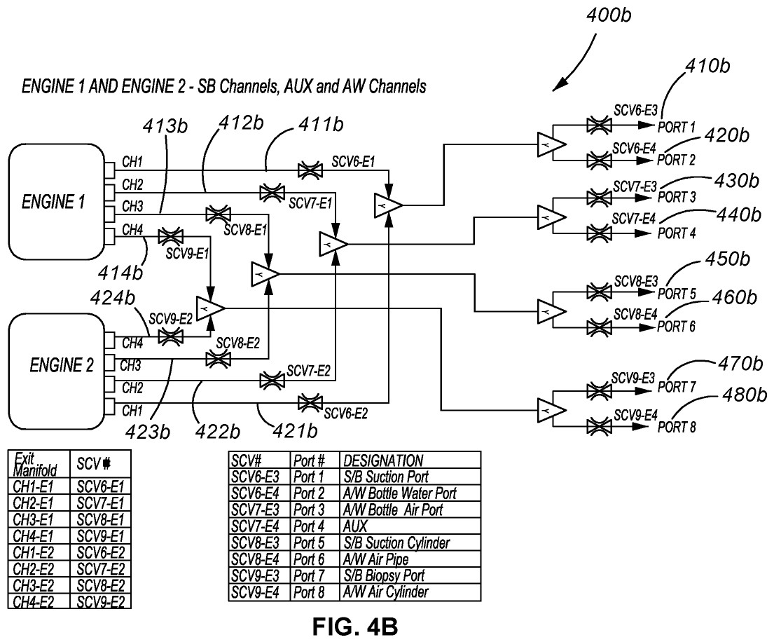

FIG. 3A shows a block diagram one embodiment of cleaning system with an integrated fluid-based powder conveyance subsystem 300a that employs 4 engines to clean multiple lumens, e.g., multiple channels of an endoscope and/or multiple lumens of multiple endoscopes, by alternating between cleaning agent, e.g., sodium bicarbonate, dosing and cleaning steps. For example, the method steps may be repeated such that the method alternates between dosing and cleaning steps to increase the efficiency of the cleaning systems and methods. In one embodiment, after warm up step 310a, engines 1 and 3 are configured to perform leak test 320a (e.g., one or two leak tests). Leak tests can be performed by flowing air or water through the medical device and measuring the water pressure and/or flow rate. A leak can be detected by low pressure and/or high flow rate. In some embodiments, when a leak is detected, an error message may be conveyed to the user and/or flow of the test fluid may be automatically stopped. The error message can inform the user whether the connections between each medical device port and the cleaning system with an integrated fluid-based powder conveyance subsystem channels correct (e.g., as shown in FIG. 4A) or are leaking, or whether the medical device has a blockage, tear, or other fault. If not errors/faults are detected, engines 1 and 3 are further configured to perform pre flush 330a (e.g., one, two, or three pre-flushes with water). Then engines 2 and 4 are configured to perform dose step 340a and clean step 350a (e.g., one, two, or three cleaning steps). The dose step includes dosing/delivering a portion (or the target dosage) of cleaning agent to an eductor. Depending on the cleaning algorithm, surfactant may also be dosed to the eductor. The inventors observed improved cleaning when surfactant was included in every other cleaning cycle than when surfactant was included in all cleaning cycles. Surfactant can be included in one of every two, three, four, or five cleaning cycles, or combinations thereof. These steps may be repeated, e.g., for about 10, about 15, about 20, about 25, about 30, about 35, or about 40 cycles. Engines 2 and 4 are also configured to perform post-flush step 360a (e.g., one, two, or three post-flushes with water), purge step 370a (e.g., one, two, or three purges with air), and end 380a.

FIG. 3B shows a block diagram another embodiment of cleaning system with an integrated fluid-based powder conveyance subsystem 300b that employs 2 engines to clean multiple lumens, e.g., multiple channels of an endoscope and/or multiple lumens of multiple endoscopes, by alternating between cleaning agent, e.g., sodium bicarbonate, dosing and cleaning steps. For example, the method steps may be repeated such that the method alternates between cleaning agent, water, and optional surfactant dosing and cleaning steps to increase the efficiency of the cleaning systems and methods as described above with respect to FIG. 3A. In one embodiment, after input check 310b verifies whether each channel of cleaning system with an integrated fluid-based powder conveyance subsystem 300b is attached to the expected medical device port (e.g., as shown in FIG. 4B), leak test 320b is performed to determine whether there are any leaks in the connections between the cleaning system with an integrated fluid-based powder conveyance subsystem channels and the medical device ports. Leak test 320b can also detect whether the medical device has faults, such as holes/tears, that would cause a leak. Block detection test 325b determines whether there are any obstructions in the lumen(s)/passage(s) of the medical device. Input check 310b, leak test 320b, and block detection test 325b may be performed by flowing air, water, or another fluid through cleaning system with an integrated fluid-based powder conveyance subsystem 300b connected to the medical device and measuring the pressure and/or flow rate and comparing those values to the expected values for the particular medical device. Such expected test values can be stored in a database for each type of medical device, model number, the respective channels/lumens, for example without limitation. If all preliminary tests clear, and the cleaning cycle begins, engine 2 performs flush 330b. Then engine 1 is configured to perform dose step 340b and clean step 350b. While engine 1 performs clean step 350b, engine 2 is configured to perform dose step 340b. While engine 2 performs clean step 350b, engine 1 performs dose step 340b, and so on. The dose step includes dosing/delivering a portion (or the target dosage) of cleaning agent to an eductor. Depending on the cleaning algorithm, surfactant may also be dosed to the eductor. The inventors observed improved cleaning when surfactant was included in every other cleaning cycle than when surfactant was included in all cleaning cycles. Surfactant can be included in one of every two, three, four, or five cleaning cycles, or combinations thereof. These steps may be repeated, e.g., for about 10, about 15, about 20, about 25, about 30, about 35, or about 40 cycles. Engine 2 is also configured to perform post-flush step 360b (e.g., with water) and purge step 370b (e.g., with air). After end 380b, maintenance step 390b can be performed.

FIG. 3C illustrates another paradigm for a two engine cleaning system that may be implemented in accordance with embodiments of the invention. In particular, FIG. 3C illustrates a cleaning process may begin with an input check. The input check may include processes such as: verifying that the supply pressures of the liquid/carrier fluid is within specification; verifying that valves to be utilized are properly configured to control the pressure to desired amounts. It is illustrated that while a first engine is ‘dosing’—e.g., while a first engine is obtaining a precise amount of sodium bicarbonate to be used for cleaning—a second engine is flushing, i.e., the second engine is flushing the target lumen to be cleaned with a fluid (e.g., water and/or air). It is illustrated that the first engine can then clean the target lumen (e.g., as described above), while the second engine can begin the dosing process. Notably, it is illustrated that this part of the cleaning cycle can implement surfactant. It is illustrated that subsequent to this period, the first and second engines invert their functions—e.g., such that the second engine is now cleaning the lumen and the first engine is dosing. This pattern can repeat any number of times to clean a target lumen. Subsequent to these cycles, the lumen can be flushed (e.g., with air and/or water) as illustrated. And subsequent to this flushing, the lumen can be purged, e.g. using air at 30 psi. The purging can have the effect of removing residual gross water.

While several examples have been illustrated and discussed regarding how a plurality of cleaning engines can be implemented to efficiently clean a target lumen. It should be appreciated that a plurality of cleaning engines can be implemented in any of a variety of ways to synergistically efficiently clean a target lumen.

FIG. 4A shows a block diagram of an exemplary embodiment of cleaning system with an integrated fluid-based powder conveyance subsystem 400a, in which engines 1 and 2 are configured to connect with endoscope ports, e.g., the endoscope air/water ports, and engines 3 and 4 are configured to connect with other endoscope ports, e.g., the suction, biopsy and/or auxiliary endoscope ports. Channels 411a and 421a combine and connect to port 1 410a. Channels 412a and 422a combine and connect to port 2 420a. Channels 413a and 423a combine and connect to port 3 430a. Channels 414a and 424a combine and connect to port 4 440a. In the illustrated embodiment, ports 1, 3 and 4 may be connected to a low impedance (e.g. w/ a relatively larger diameter—e.g., a suction biopsy port of an endoscope) and port 2 may be connected to a high impedance line (e.g. having a relatively smaller diameter—e.g., an auxiliary port of an endoscope). The effect of this configuration is that you can clean a combination of low impedance line(s) and high impedance line(s) in an intelligent way. This configuration can allow the aggregate to flow through the line at a relatively high velocity consistent with the capacity of the line. Thus, for example, port 2 may act as a relief line for excess cleaning aggregate (at the same time, the cleaning aggregate may operate to clean port 2). As can be appreciated, while one configuration for allowing one channel to act as a relief line has been illustrated, this concept can be implemented in any of a variety of ways in accordance with embodiments of the invention.

FIG. 4B shows a block diagram of another embodiment of cleaning system with an integrated fluid-based powder conveyance subsystem 400b, in which engines 1 and 2 are configured to connect with endoscope ports, e.g., the endoscope air/water, suction, biopsy and auxiliary endoscope ports. Channels 411b and 421b combine and connect to port 1 410b and port 2 420b. Channels 412b and 422b combine and connect to port 3 430b and port 4 440b. Channels 413b and 423b combine and connect to port 5 450b and port 6 460b. Channels 414b and 424b combine and connect to port 7 470b and port 8 480b. In one embodiment, port 1 410b is designated for the suction biopsy port, port 2 420b is designated for the water port, port 3 430b is designated for the air port, port 4 440b is designated for the aux port, port 5 450b is designated for the suction cylinder, port 6 460b is designated for the air pipe, port 7 470b is designated for the biopsy port, and port 8 480b is designated for the air cylinder. Thus, for example, ports 5 and 6 (corresponding with the suction/biopsy line and the air-water line) can be cleaned simultaneously. Here, the suction-biopsy line (low impedance) can act as a relief line for the cleaning of the air-water line (high impedance). As before, the ‘relief line’ can be cleaned at the same time. In this way, a relief line can be implemented in an efficient way.

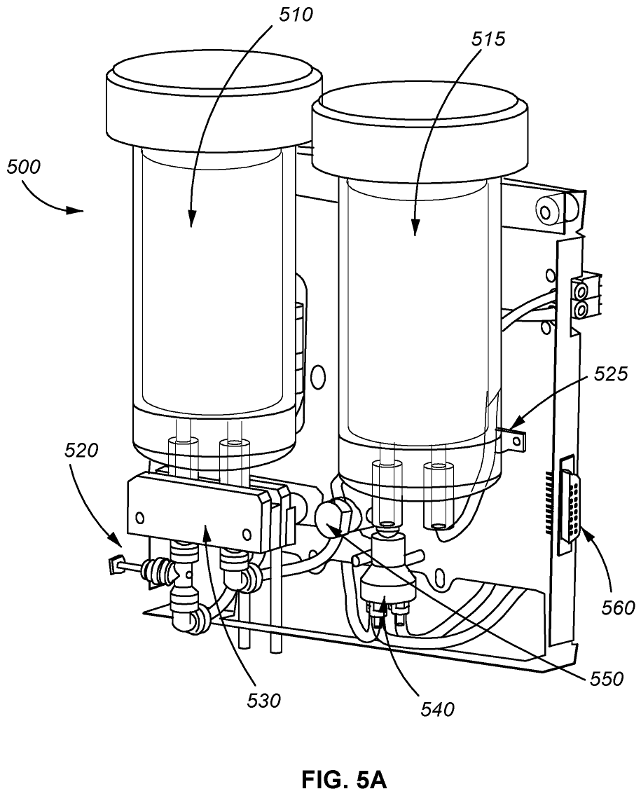

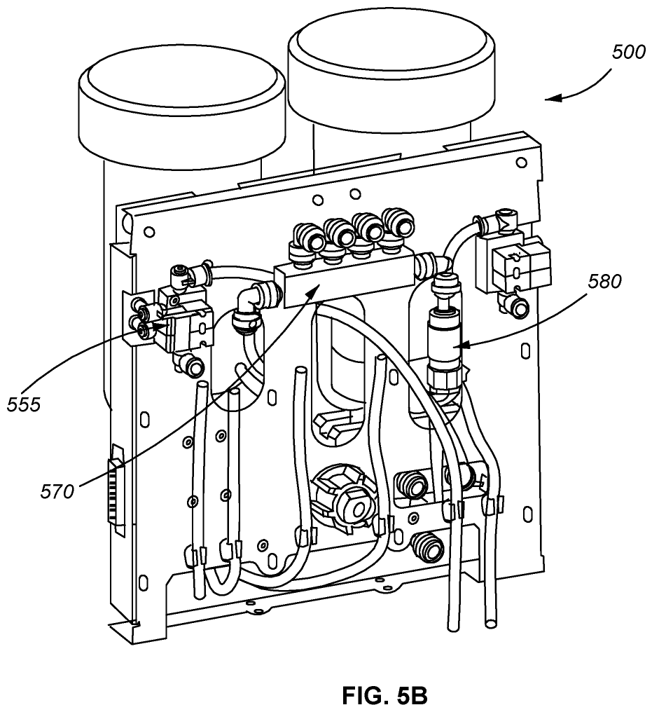

As illustrated in FIG. 5A, one embodiment of a consumable interface module 500 comprises cleaning agent/powder receiver 510 (e.g., for cleaning agents, such as sodium bicarbonate), surfactant receiver 515, pressure sensor 520, surfactant low level sensor 525, receiver interface 530, 1×4 Y-connector 540, pinch valve 550, and I/O interface 560. Exemplary consumable interface module 500 also comprises 3/2 way solenoid control valve 555, 2×4 manifold 570, and air filter 580 (FIG. 5B). Pressure sensor 520 can provide feedback on the pressure the bicarb receiver is set at. It can be used to determine if the receiver is pressurized properly. Low level sensor 525 can be used to communicate the levels of cleaning agent/powder and surfactant, respectively, to the control module, which can present the user with a message informing the user of the consumable level and/or instructing the user to refill cleaning agent/powder receiver 510 and/or surfactant receiver 515.

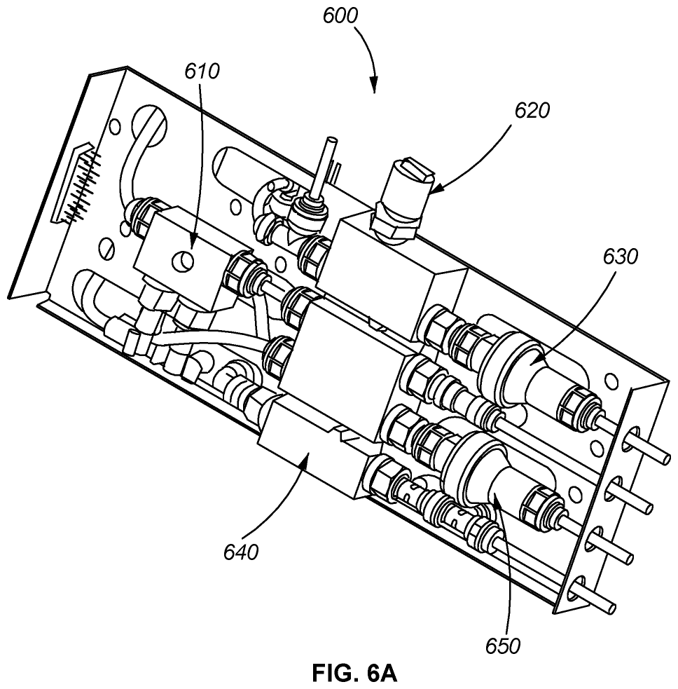

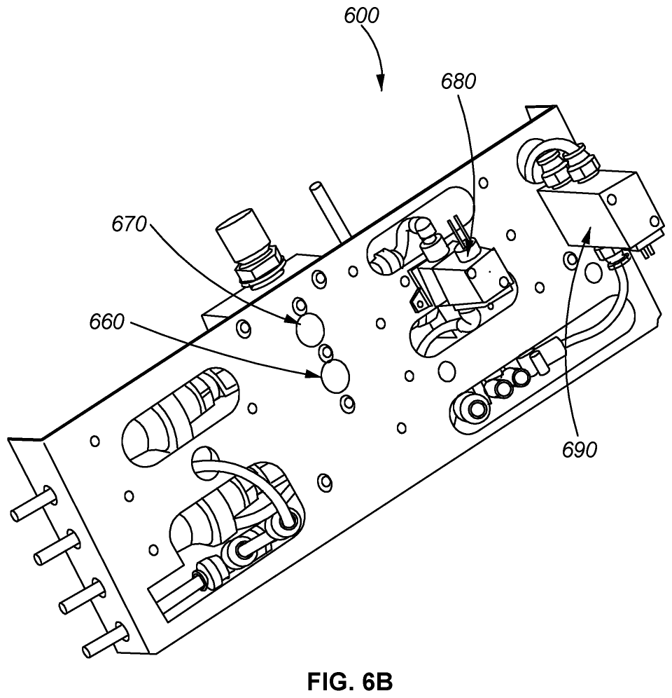

FIGS. 6A and 6B illustrate one embodiment of intake manifold module 600. Intake manifold module 600 comprises N/C—N/O air block 610, water thermocouple 620, water pressure regulator 630, intake manifold 640, air pressure regulator 650, air pressure sensor 660, water pressure sensor 670, 2-way valve 680, and 3/2-way valve 690. Intake manifold module 600 is configured to receive compressed air and control the flow and pressure of air to cleaning agent/powder receiver 510 of consumable interface module 500 and the SCS engine. Intake manifold 600 is also configured to receive water and control the flow and pressure of water to the SCS engine.

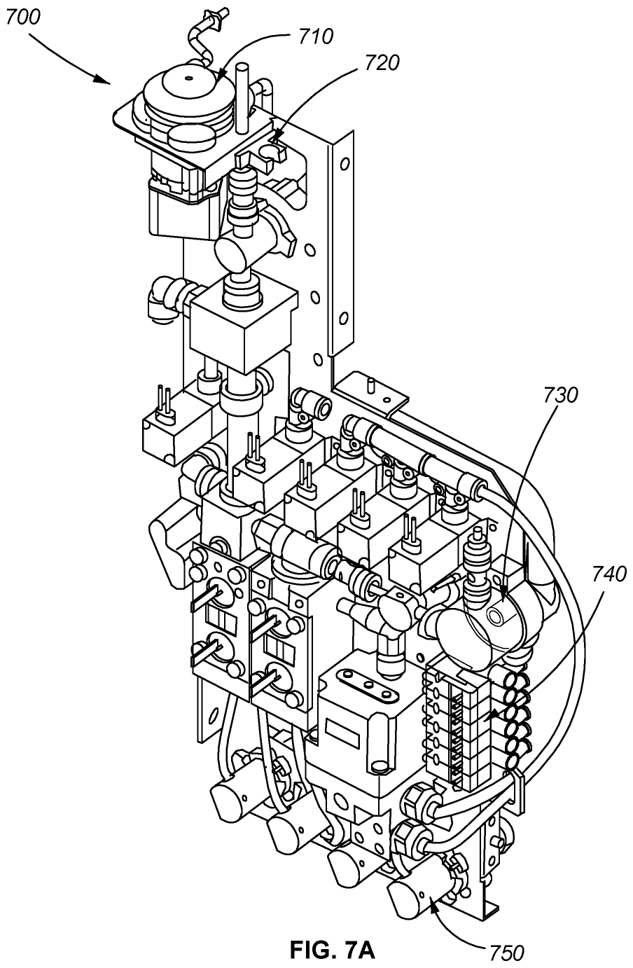

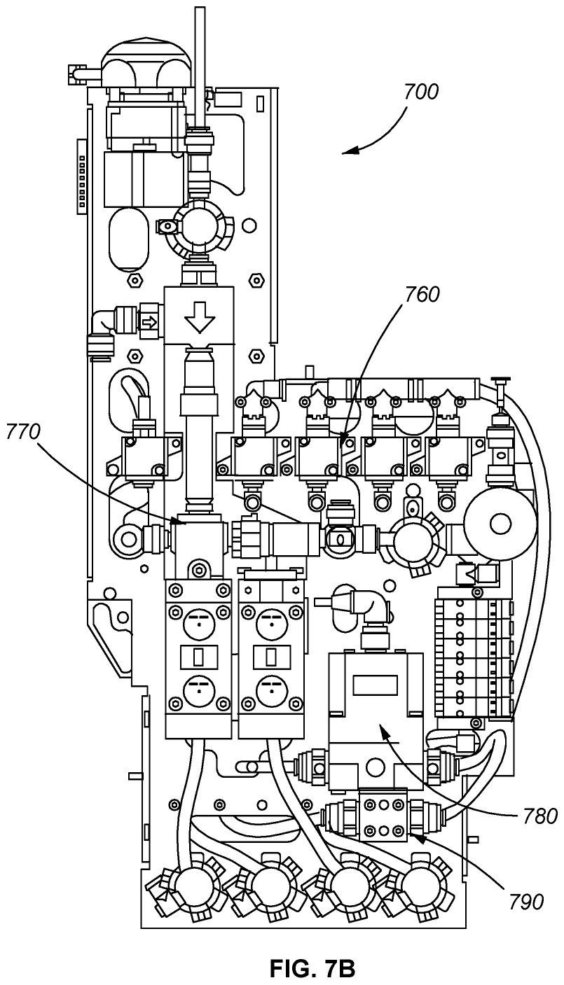

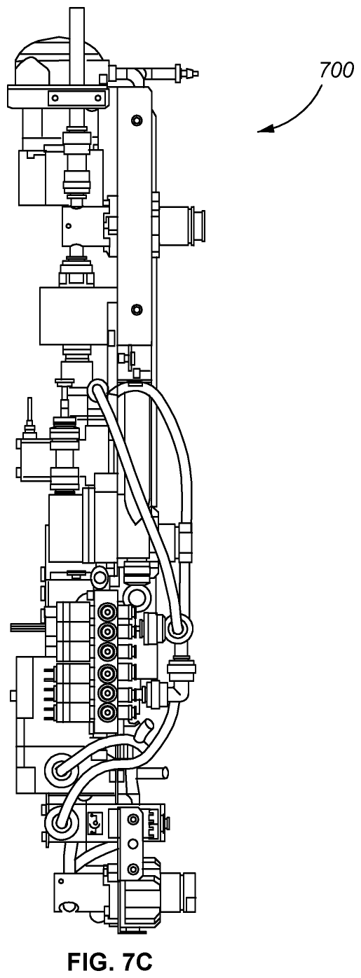

FIGS. 7A-7C illustrate one embodiment of engine assembly 700. Engine assembly 700 comprises surfactant pump 710, fluid detection sensor 720 (e.g., optical), douser vent assembly 730, solenoid control valve array 740, and pinch valve(s) 750 (FIG. 7A). Engine assembly 700 further comprises air/water solenoid valve array 760, eductor assembly 770, electronic air pressure regulator 780, and water flow control valve 790 (FIG. 7B). FIG. 7C provides a side view of engine assembly 700.

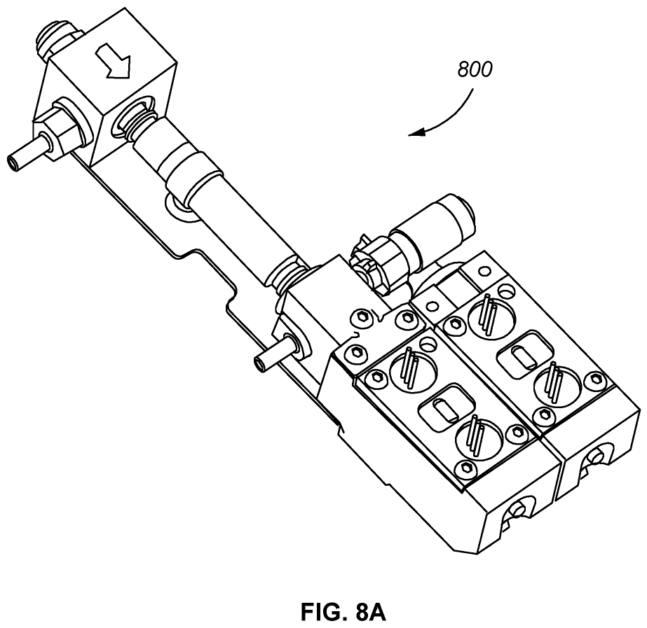

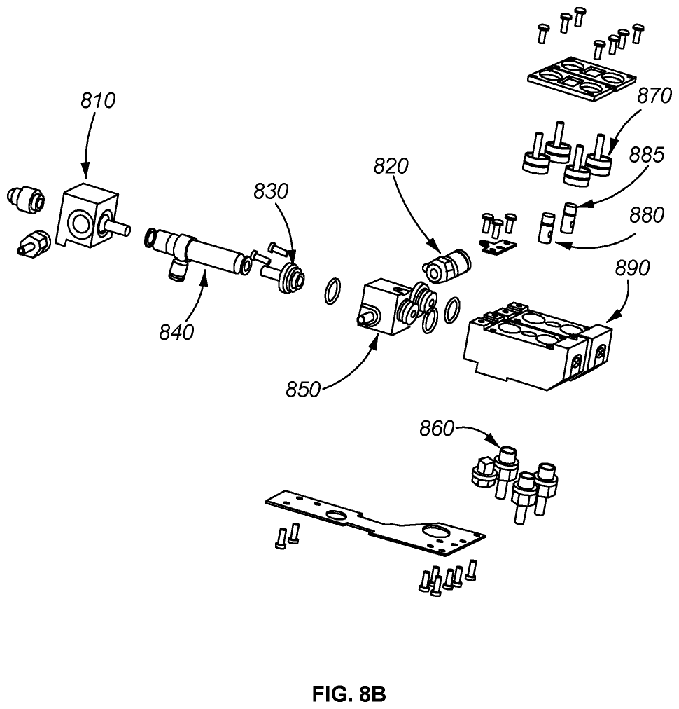

Advantageously, eductor assembly 800 can achieve on-demand cleaning agent dosing with high accuracy and precision. FIGS. 8A and 8B illustrate one embodiment of eductor assembly 800, which comprises feeder manifold 810, vent receiver/douser 820, eductor insert 830, vacuum generator 840, eductor body 850, over pressure relief valve 860, pressure sensor 870, low-z orifice 880, high-z orifice 885, and exit manifolds 890.

In the illustrated embodiment, pressure can be used to deliver cleaning agent from receiver 510 through feeder manifold 810 to the dosing filter, which sits within the vent receiver/douser 820. The pressure differential between the inlet and the outlet of the dosing filter is what drives the delivery of the cleaning agent to the dosing filter. As the dosing filter becomes increasingly blocked with cleaning agent, the pressure differential increases. Once the pressure differential increases to a known target, e.g., about 20 psi or about 30 psi, the delivery of cleaning agent to the engine is stopped. Vacuum generator 840 can generate a vacuum to clear the path of cleaning agent, which is then staged in eductor body 850, and air/water solenoid valve array 760 can open to allow water to flow into eductor body 850 with the cleaning agent, and a cleaning mixture can thereby be created. Simultaneously, a gate (e.g., pinch valve 750) to the endoscope lumen(s) can be opened, and the cleaning agent, water, air, and optional surfactant are dynamically mixed/combined/aggregated in the eductor and delivered to the endoscope. Air can be used as a carrier fluid to carry the cleaning mixture through the lumen. Depending on the fluid dynamic parameters fluid flow may or may not be turbulent.

Thus, by simply regulating pressure and flow, the cleaning system prepares cleaning mixture on-demand and alternates between dosing and cleaning cycles to quickly and effectively clean the lumens of medical devices. Periodically a surfactant can be introduced into the cleaning mixture, e.g., every other cleaning cycle, every two cleaning cycles, or every 3 cleaning cycles. Surprisingly, better cleaning is achieved when mixtures comprising surfactant are alternated with mixtures without surfactant than when surfactant is included in every cleaning mixture. However, including surfactant in every mixture is not excluded. A further advantage of the disclosed systems is that by maintaining positive air pressure, water is prevented from flowing back up to the consumable interface.

The use of the dosing filter allows delivery of accurate and precise amounts of cleaning agent to the engine. Although pressure differential-based systems and methods disclosed herein result in highly accurate and precise amounts of cleaning agent, time-based systems and methods are also contemplated. It should be appreciated that pressure differential measurements can also be used to track the life of the filter. For example, with use the filter may swell or degrade filter, and such changes in the filter may be monitored and/or detected using a control algorithm. A further advantage of the disclosed systems and methods is that high air pressures are not required, so the systems can be used in settings where high air pressure is not available, e.g., kitchen appliances and medical devices.

The ability to prepare accurate and precise mixtures on-demand confers numerous advantages, including addressing the challenge of cleaning endoscope having intricate flow paths. A challenge posed by small lumens is their high resistance to fluid flow, using smaller mixture portions allows those portions to achieve higher velocities and better cleaning in such lumens. Additionally, narrow passages and nozzles may have diameters only 0.3 mm across, and if the amount of cleaning agent is not controlled, the narrow passages and nozzles may clog. The use smaller portions of mixture over multiple cycles solves the problem of blocking nozzles. The use of smaller chambers can make it easier to control the amount of cleaning agent included in cleaning mixtures. Additionally, the use of smaller chambers permits very fine pressure control, yielding improved reliability and repeatability, whereas in larger vessels, pressure can accumulate and may cause blow-outs.

A further advantage of fluid-based powder cleaning agent transport is that the consumable reservoirs do not need to be proximal to the engine itself and can be located in an array of positions, which provides the ability to locate in a position that is easy for an end user to access.

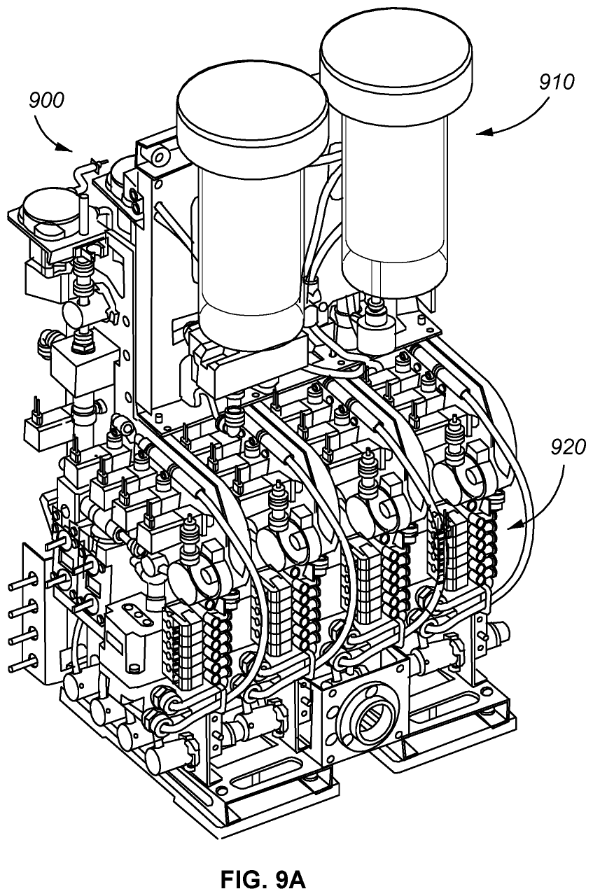

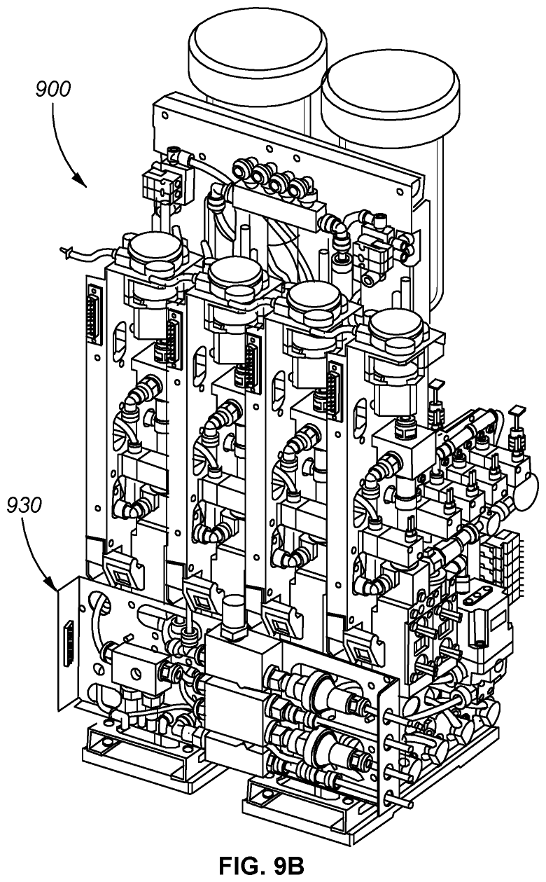

FIGS. 9A and 9B illustrate one embodiment of system 900 for cleaning the lumens of medical devices comprising consumable interface module 910, 2×2 engine assembly 920, and intake manifold module 930.

The disclosure further provides a method of cleaning a lumen of a medical device comprises providing a device for cleaning endoscopes configured to: mix a first portion of cleaning agent, e.g., sodium bicarbonate, and a first portion of water to form a mixture/combination/aggregate; inject a portion of the mixture into the lumen of the medical device; inject a quantity of air into the lumen of the medical device with the portion of the mixture.

Therefore one embodiment of a system for cleaning a medical device having a lumen comprises: an eductor comprising a filter; a fluid-based powder delivery subsystem configured to deliver a cleaning agent to the eductor and comprising a pressure differential mechanism, wherein the pressure differential mechanism is configured to deliver fluid-based powder cleaning agent (e.g., pneumatically) to the eductor when the pressure differential is less than a threshold value; a liquid delivery subsystem configured to deliver the liquid to the eductor; and an engine configured to dynamically mix/combine/aggregate the cleaning agent and the liquid and propel the resulting mixture through a lumen of a medical device.

It should be appreciated that functions may be performed by any suitable component including one or more parts/modules disclosed herein. Table 1 provides the modules and assemblies of an exemplary endoscope cleaning system and their corresponding functions.

TABLE 1
FUNCTIONS PERFORMED BY THE MODULES AND ASSEMBLIES OF AN
EXEMPLARY ENDOSCOPE CLEANING SYSTEM.

| | | Intake | Consumable | | | | |
|---|---|---|---|---|---|---|---|
| Function | Control | Manifold | Interface | SCS | Coupling | Adaptor | Endo- |
| Performed | Module | Module | Module | Engine | Assembly | Assembly | scope |
| WARM UP | X | X | | | | | |
| LOW LEVEL | X | X | X | X | | | |
| CONSUMABLE | | | | | | | |
| DETECTION | | | | | | | |
| LEAK AND | X | X | | X | X | X | X |
| BLOCKAGE | | | | | | | |
| DETECTION | | | | | | | |
| PRE-FLUSHING | X | X | | X | X | X | X |
| DOSING | X | X | X | X | X | X | |
| CLEANING | X | X | X | X | X | X | X |
| POST FLUSH | X | X | | X | X | X | X |
| PURGE | X | X | | X | X | X | X |
| DEVICE SELF | X | X | X | X | | | |
| MAINTENANCE | | | | | | | |

It should also be appreciated that cleaning efficiency can be modulated using multiple parameters, including without limitation, the number of shots of cleaning mixture, surfactant pump feed rate, water temperature, water pressure, and air pressure.

### Consumable Interface Modules

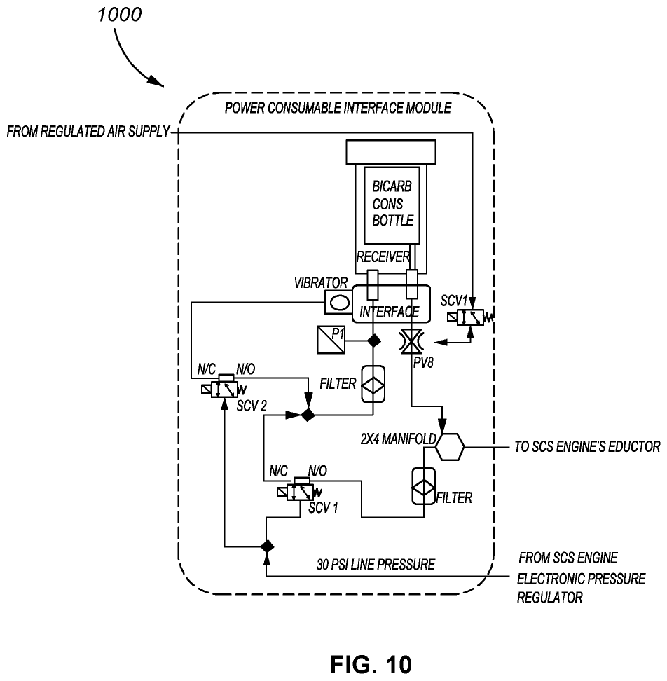

Certain embodiments of the invention relate to consumable interface modules that may be implemented in connection with the above described systems/methodologies. FIG. 10 illustrates a consumable interface module that may be implemented in accordance with embodiments of the invention. In this context, ‘consumable’ characterizes the cleaning agents (e.g. sodium bicarbonate) and/or surfactant that may be utilized by cleaning systems. In the illustrated embodiment, a positive conveying system can deliver the cleaning agent to the cleaning engine. In some embodiments, an alternating pulse supply method may be implemented. For example, an alternating pulse supply method can use two valves (e.g. SCV1 and SCV2) to alternately pulse the air supply to the receiver. The pulsing can occur at 4 Hz for example. This can help quickly deliver the cleaning agent (especially, when it is at a low level). In the illustrated embodiment, it is shown that vibration agitation (e.g. using a pneumatic vibrator) may be used to manage powder bridging. It can be appreciated that other mechanisms to implement agitation can be similarly used. In some embodiments, there is no agitating mechanism. In the illustrated embodiment, Powder/Bicarb Consumable Bottle is loaded to the Receiver, is then pressurized to 30 psi, vibrated for target pulse frequency, and an alternate pulse supply method may be implemented until the differential pressure reach the target differential pressure value. Of course, consumable interface modules can be implemented using a variety of techniques and architectures in accordance with embodiments of the invention.

1. A method of cleaning a lumen comprising:
flowing a first fluid comprising a cleaning agent to a chamber,
wherein flowing the first fluid comprising the cleaning agent to the chamber creates a pressure differential across a filter fluidly coupled to the chamber;
reaching a threshold by the pressure differential, thereby triggering the flowing of the first fluid comprising the cleaning agent to the chamber to stop; and
flowing a second fluid through the chamber to convey the cleaning agent through a lumen, thereby cleaning the lumen.

2. The method of claim 1, wherein the first fluid comprises a first gas selected from the group consisting of air, nitrogen, argon, or carbon dioxide.

3. (canceled)

4. The method of claim 1, wherein the first fluid comprises a first liquid, and the cleaning agent is substantially insoluble in the first liquid, and wherein the first liquid comprises at least one of aqueous liquid, an alcohol, a hydrocarbon, or carbon dioxide.

5. (canceled)

6. The method of claim 1, wherein a cross-sectional area of the filter is perpendicular to a direction of the flowing first fluid.

7. The method of claim 1, wherein a cross-sectional area of the filter is parallel to a direction of the flowing first fluid.

8. The method of claim 1, wherein the second fluid comprises a second gas selected from the group consisting of air, nitrogen, argon, or carbon dioxide.

9. (canceled)

10. The method of claim 8, wherein the second fluid comprises a second liquid selected from the group consisting of an aqueous liquid, an alcohol, a hydrocarbon, or carbon dioxide.

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. The method of claim 1, wherein flowing a first fluid comprising a cleaning agent to a chamber comprises:
delivering a target dosage of the cleaning agent to an eductor, wherein the cleaning agent is pneumatically delivered through the filter to achieve the target dosage;
delivering a fluid to the eductor; and
delivering an aggregate of the fluid and the target dosage of the cleaning agent to at least a portion of the lumen.

35. The method of claim 34 further comprising repeating the steps of delivering the target dosage of the cleaning agent to the eductor, delivering the fluid to the eductor, and delivering the aggregate to clean the at least a portion of the lumen.

36. The method of claim 34 further comprising delivering a surfactant to the eductor, and wherein the aggregate further comprises the surfactant.

37. The method of claim 1, further comprising:
mixing a first portion of the cleaning agent and a first portion of water to form an aggregate;
injecting the aggregate into the lumen of the medical device; and
injecting a quantity of air into the lumen of the medical device after the aggregate.

38. The method of claim 37, wherein a proportion of the first portion of cleaning agent to the first portion of water is about 0.5% to about 5%, or about 1% to about 3%.

39. The method of claim 1, wherein the cleaning agent comprises sodium bicarbonate.

40. The method of claim 37, wherein the first portion of cleaning agent is about 1 g to about 10 g or about 4 g to about 6 g.

41. The method of claim 37, wherein the first portion of water is about 50 g to about 500 g or about 100 g to about 400 g.

42. The method of claim 37, wherein the first portion of cleaning agent further comprises air.

43. The method of claim 37, further comprising a step of heating the water to a temperature before the mixing/combining/aggregating step.

44. The method of claim 43, wherein the temperature is about 15° C. to about 25° C. or about 25° C. to about 40° C.

45. The method of claim 37, wherein the mixing comprises mixing a first portion of surfactant and the first portion of cleaning agent and the first portion of water to form the aggregate.

46. The method of claim 45, wherein the first portion of surfactant is about 0.1 g to about 3 g or about 0.5 g to about 1.5 g.

47. The method of claim 37, wherein a flow of the mixture/combination/aggregate is turbulent.

48. (canceled)

49. (canceled)

50. (canceled)
